(19) United States
(12) Patent Application Publication
(10) Pub. No.: US 2007/0031468 A1
(43) Pub. Date: Feb. 8, 2007

(54) Modified chitosan for vascular embolization

(75) Inventors: John M. Abrahams, Scarsdale, NY (US); Weiliam Chen, Mount Sinai, NY (US)

Correspondence Address: SCHWEGMAN, LUNDBERG, WOESSNER & KLUTH, P.A., P.O. BOX 2938, MINNEAPOLIS, MN, 55402, US

(73) Assignee: Endomedix, Inc.

(21) Appl. No.: 11/447,794

(22) Filed: Jun. 6, 2006

Related U.S. Application Data
(60) Provisional application No. 60/705,319, filed on Aug. 4, 2005.

(51) Int. Cl. A61F 2/00

(52) U.S. Cl. 424/423

(57) ABSTRACT

A therapeutic composition and a method are provided for occlusion of a vascular site. The vascular site may be within a blood vessel or a lymph duct, and may include an aneurysm, an arteriovenous malformation. The composition comprises an acrylated chitosan dissolved in an aqueous medium. The composition is preferably a flowable liquid at a pH of about 6-6.5, and gels or solidifies in situ in the vascular site at a physiological pH of about 6.9-7.4. The method comprises introducing the composition to the interior of the vascular site, as with a catheter. The composition may further include a bioactive agent or intact cells, or a radiopaque agent.

### CLAIM OF PRIORITY FROM A PRIOR-FILED PROVISIONAL APPLICATION

This application claims the benefit of priority, under 35 U.S.C. Section 119(e), to U.S. Provisional Patent Application No. 60/705,319, filed on Aug. 4, 2005, which is incorporated herein by reference.

### GOVERNMENT GRANT SUPPORT

This invention was made with the support of the National Institutes of Health under grant no. DK068401 and HL65175. The U.S. Government has certain rights in the invention.

### BACKGROUND

The deliberate embolization of vascular ducts, such as blood vessels or lymph ducts, that is, the deliberate endovascular partial or complete obstruction or occlusion of blood vessels or lymph ducts, is a useful therapeutic process that can be employed in a number of clinical situations. For example, endovascular embolization has been used to control vascular bleeding, to reduce the blood supply to tumors, and to occlude vascular aneurysms, particularly intracranial aneurysms. In recent years, endovascular embolization for the treatment of aneurysms has received much attention.

An aneurysm is a localized dilation of a blood vessel that represents a malcondition with potentially fatal consequences. In an aneurysm, under the pressure exerted by the blood stream, a weakened section of the vessel wall balloons out in excess of the normal diameter of the vessel. Aneurysms can occur in various forms, but all share the feature of a stretched, weakened blood vessel wall. Such a stretched, weakened section of the vessel has an increased probability of rupture, which can result in hemorrhagic stroke if the vessel is within the brain, and can cause potentially life-threatening internal bleeding, especially if the aneurysm is situated in a major artery such as the aorta. Cerebral arteries, such as those making up the circle of Willis, are one of the most common sites for aneurysms, and the rupture of an aneurysm in this location carries a very high risk of severe injury or death from subarachnoid intracerebral hemorrhage.

The wall of a blood vessel is considered to comprise three major layers: the intima (the innermost layer) that is in contact with the blood, formed largely of endothelial cells; the tunica media (middle layer), formed of smooth muscle; and the adventitia (outer layer), formed of connective tissue. A true aneurysm involves the stretching of all three layers. In the development of an aneurysm, an already weakened locus in the blood vessel wall becomes increasingly more vulnerable to further stretching and expansion, leading to an even weaker section of vessel wall. This phenomenon is described by the Laplace Law, which provides that the arterial wall tension is a function of the product of blood pressure and vessel diameter at a given vascular location. As the diameter increases, wall tension increases, possibly resulting in eventual rupture. Also, the aneurysm site is known to breed thrombi, blood clots within the blood stream, that can detach and drift downstream until they encounter a vessel of insufficient diameter, where they can cause a blockage with potentially damaging or fatal consequences.

Endovascular thrombogenic microcoils are gradually becoming the standard of treatment for intracranial aneurysms, including for most posterior circulation and some anterior circulation aneurysms. Although there are numerous variations of the general technology, most are dependent on platinum microcoils of assorted shapes that detach through an electrolytic reaction for deployment in the aneurysm sac. They are typically introduced into the brain vasculature via the femoral artery. Once deployed, microcoils induce arterial stasis within the dome, clot formation and occlusion, and eventual fibrosis with obliteration usually within 12 months. However, despite initial successes, there are pitfalls with this treatment modality. For instance, wide-neck and larger aneurysms are not as effectively treated with traditional endovascular methods, often requiring repeat coiling procedures.¹⁻⁴ Moreover, the most optimal geometry for coiling is when the neck is less than half the size of the dome or when the neck is less than 4 mm.

Previous reports demonstrated that biodegradable polymer (poly-lactide-co-glycolide) coated platinum coil could achieve accelerated fibrosis and obliteration by intensifying aneurismal neointimal formation in animal models.⁵⁻⁶ Other surface modifications include directed cellular responses, ion impingement, and protein coating, aimed to modulate the coil surface properties for complete aneurysm obliteration.⁷⁻²¹ Others have shown that a range of proteins coated onto the coil surface such as albumin, collagen, fibronectin and vascular endothelial growth factor can produce favorable biological responses.^{7-14, 22}

The rate and extent of thrombosis depends on a number of factors including coil composition, packing density, surface charge density, surface texture, and extent of intimal injury.²³ However, coil embolization does not reinforce the weakened blood vessel wall and does not always result in replacement of the aneurysm thrombus with tissue.²⁴ In addition, the long-term consequence of permanently deploying these non-degradable coils into the cerebral vasculature is not known.

The optimal clinical goal of coil embolization in an aneurysm is to induce stasis, thrombosis leading to fibrotic tissue formation, and eventually endothelialization across the aneurysm orifice. However, histopathological evaluation of human aneurysm specimens implanted with platinum microcoils suggested the presence of unorganized clot and fluid spaces between the coils and the aneurysm.²⁵⁻³¹ Even though packing aneurysms with platinum coils appear to increase their stability through thrombosis, due to its relative bio-inertness, platinum contributes little stimulus to fibrotic tissue formation.

Another approach is the direct injection of a liquid polymer embolic agent into the vascular site to be occluded. One type of liquid polymer used in the direct injection technique is a rapidly polymerizing liquid, such as a cyanoacrylate, particularly isobutyl cyanoacrylate, that is delivered to the target site as a liquid, and then is polymerized in situ. Alternatively, a liquid polymer that is precipitated at the target site from a carrier solution has been used. An example of this type of embolic agent is a cellulose acetate polymer mixed with bismuth trioxide and dissolved in dimethyl sulfoxide (DMSO). Another type is ethylene glycol copolymer dissolved in DMSO. On contact with blood, the DMSO diffuses out of the vessel, and the polymer precipitates and rapidly hardens into an embolic mass that can conform to the shape of the aneurysm. Other examples of materials used in this “direct injection” method are disclosed in the following U.S. Pat. No. 4,551,132-Pasztor et al.; U.S. Pat. No. 4,795,741-Leshchiner et al.; U.S. Pat. No. 5,525,334-Ito et al.; and U.S. Pat. No. 5,580,568-Greff et al. Still another approach to the chemical embolization of an abnormal vascular site is the injection into the site of a biocompatible hydrogel, such as poly(2-hydroxyethylmethacrylate) (“pHEMA” or “PHEMA”); or a polyvinyl alcohol foam (“PAF”). See, e.g., Horák et al., “Hydrogels in Endovascular Embolization. II. Clinical Use of Spherical Particles”, Biomaterials, 7, 467 (November, 1986); Rao et al., “Hydrolysed Microspheres from Cross-Linked Polymethyl Methacrylate”, J. Neuroradiol., 18, 61 (1991); Latchaw et al., “Polyvinyl Foam Embolization of Vascular and Neoplastic Lesions of the Head, Neck, and Spine”, Radiology, 131, 669 (June 1979). These materials are delivered as microparticles in a carrier fluid that is injected into the vascular site, a process that has proven difficult to control. Ken (U.S. Pat. No. 6,113,629) has generally disclosed occluding the necks of aneurysms with hydrogels that cross-link and solidify upon exposure to body temperatures. The hydrogel can be used as a carrier for growth factors and a radiopaque agent. However, a continuing need exists for effective, controllable, non-mechanical treatments for aneurysms and other vascular abnormalities requiring repair and/or stabilization.

### SUMMARY

The present invention provides a therapeutic composition and a therapeutic method useful for embolizing, that is, for partially or completely occluding, a endovascular site having a defined interior shape and volume, such as an aneurysm or other arteriovenous malformation. The composition and the method are also useful for embolizing a section of normal blood vessel for the purpose of occluding the vessel as may be desirable in treatment of a tumor that is vascularized by the blood vessel, or to control downstream bleeding from the blood vessel. The composition and the method can also be used for embolization of other vascular ducts, such as lymph ducts, when such therapy is indicated, such as for repair of a lymphatic leak due to trauma, surgery, or disease.

The composition of the present invention comprises a flowable aqueous solution of an acrylated chitosan, adjusted to a slightly acidic pH (preferably about 6.0-6.5) such that the acrylated chitosan solution remains a flowable liquid under ambient conditions (i.e., about 20-25° C.) at least for a sufficient period of time for it to be prepared and introduced into a blood vessel or lymph duct. Upon contact with an aqueous medium at near-neutral or slightly alkaline pH such as exists in living human tissue fluids, such as blood or lymph, which have a physiological pH of about 6.9-7.4, the composition of the invention solidifies or gels into a hydrogel that totally or partially fills the vascular target site.

The method comprises introducing the composition comprising the flowable aqueous acrylated chitosan solution endovascularly so that the acrylated chitosan solution solidifies or gels in situ to occlude the interior volume of the aneurysm or other arteriovenous malformation, or a section of a normal blood vessel or lymph duct. This flowable aqueous solution may be introduced at the site through a catheter inserted to the vessel or duct.

The composition can also include a dissolved or dispersed radiopaque agent, allowing the composition of the invention to be visualized during and after emplacement using standard angiography techniques.

The invention further provides therapeutic combinations comprising the composition of the invention and bioactive agents including living cells such as regenerative cells, as well as recombinant DNA; cytokines, including growth factors such as fibroblast growth factor (FGF) or vascular endothelial growth factor (VEGF); inflammatory agents, anti-inflammatory agents, immunomodulatory agents; or radioactive particles or complexes. Matrix stabilizing agents such as cytochalasin B can also be included. When the agent comprises a polypeptide, heparin or a bioactive fragment or derivative thereof can be mixed with the composition to further stabilize the polypeptide against degradation.

A feature of the present invention is that the near-neutral pH of the flowable aqueous acrylated chitosan solution prior to emplacement within the blood vessel allows for such agents and intact cells to survive substantially undegraded and, in the case of cells, to remain viable. This is in contrast to the much more acidic pH that is needed to solubilize underivatized chitosan which would tend to cause severe degradation of the added agent or death of the cells.

The therapeutic combination of the composition and the bioactive agent serves to promote cellular proliferation or regeneration within the volume of the site and to eliminate and heal the abnormal site employing, at least in part, endogenous cellular processes, such as fibrosis, matrix stabilization and the like.

### BRIEF DESCRIPTION OF THE FIGURES

FIG. 1 Acrylated chitosan (aCHN) gelation in pH 7.4 phosphate buffered saline. Panel (A): initial formation of two phases; (B): gelation of aqueous aCHN phase (circled).

FIG. 2 Schematic illustration of the surgical procedure required for polymer gel infusion. (A) Placement of the permanent distal ligature and temporary proximal ligature on the exposed common carotid artery. (B) Release of the temporary ligature after infusion of polymer gel, another permanent ligature is placed next to the arteriotomy site for closure. There was noticeable dilation of the artery after polymer gel infusion.

FIG. 3 The extent of occlusion of artery two weeks after intervention. (1) Pristine arteries, (2) arteries infused with aCHN polymer gel, (3) arteries infused with saline, (4) arteries infused with VEGF solution, and (5) arteries infused with bioactive VEGF/aCHN polymer gel. The p-values in the figure represent the statistical difference between individual treatment and the arteries receiving VEGF/aCHN polymer gel.

FIG. 4 Representative hematoxylin and eosin stained histological specimens of the Common Carotid Arteries 2 weeks after intervention. (A) VEGF/aCHN polymer gel, (B) aCHN polymer gel only, (C) saline, and (D) VEGF solution.

### DETAILED DESCRIPTION OF PREFERRED EMBODIMENTS

As used herein, the term “vascular system” refers to the system of vessels and tissues that carry or circulate fluids such as blood or lymph throughout a living mammalian body. The term “vascular” means of or pertaining to the vascular system. A “vascular site” is a discrete location within the vascular system or a relatively small section of a vascular vessel or duct.

The term “embolize” as used herein refers to obstructing or occluding a volume of a vascular site, either partially or completely, through emplacement of an embolus. When occlusion is complete, fluid flow through the vessel is blocked, whereas partial occlusion allows for fluid flow past the embolus.

As used herein, a “vascular occlusive composition” refers to a composition of the invention comprising an acrylated chitosan. An “effective embolic amount” of a vascular occlusive composition is an amount of the composition sufficient to cause partial or complete occlusion of a vascular vessel or duct.

An “aneurysm” is a localized, blood-filled dilation of a blood vessel.

“Intracranial circulation” means blood circulation within the cranium.

“Posterior circulation” means blood circulation in the posterior cerebral artery.

“Anterior circulation” means blood circulation in the anterior cerebral artery.

“Chitosan,” as the term is used herein, refers to deacetylated chitin, the natural product found in fungi and crustacean shells. Chitosan is polymeric D-glucosamine (2-amino-2-deoxyglucose) linked in the β-1,4 configuration.

An example of a section of a chitosan chain has the following chemical structure, wherein the number of glucosamine units may range from only a few upwards into the hundreds:

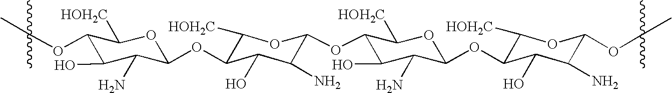

Chitosan is commercially available in a wide range of purities, degrees of polymerization, and degrees of deacetylation, from a number of suppliers. It is biocompatible and biodegradable, and has been used to form films, in biomedical devices and to form microcapsule implants for controlled release in drug delivery. See, e.g., S. Hirano et al., Biochem. Sys. Ecol., 19, 379 (1991); A. D. Sezer, Microencapsulation, 16, 687 (1999); A. Bartkowiak et al., Chem. Mater. 11., 2486 (1999); T. Suzuki et al., Biosci. Bioeng., 88, 194 (1999). Chitosan provides a non-protein matrix for 3-dimensional tissue growth, and activates macrophages for tumoricidal activity. It stimulates cell proliferation and historarchitectural tissue organization. Chitosan is a hemostat, which assists blood clotting and blocks nerve endings reducing pain. Chitosan will gradually depolymerize to release β-D-glucosamine, which initiates fibroblast proliferation, helps in ordered collagen deposition and stimulates increased levels of natural hyaluronic acid synthesis at the vascular trauma site.

The novel vascular-occlusive composition of the invention is prepared by reaction of chitosan with acrylic acid.

An exemplary structure of a section of acrylated chitosan (aCHN) has the formula:

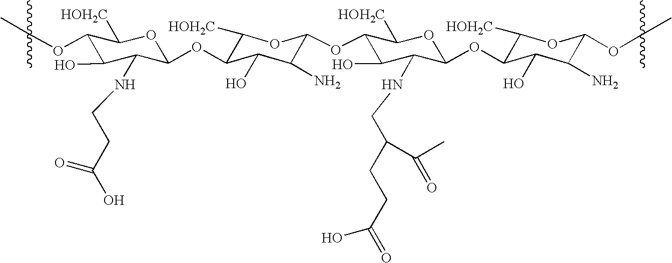

While not wishing to be bound by theory, it is believed that the acrylate moieties are bonded to the chitosan molecule via Michael addition of the chitosan amino groups to the acrylate β-carbons. As can be seen, not every monomeric unit is necessarily substituted with an acrylate moiety. The average number of acrylate moieties per monomeric aminoglucose unit (degree of substitution) may vary. Furthermore, acrylate oligomerization may occur such that more than a single acrylate unit is bonded to a given monomeric aminoglucose unit. Other structures of acrylated chitosan may be employed in the present composition without departing from the principles of the invention. The addition of acrylate moieties to chitosan serves to convert an alkaline polymer bearing amino groups to an ampholytic polymer bearing both amino groups and carboxylic acid groups. Acrylated chitosan is therefore a polymeric amino acid.

For example, to obtain aCHN, chitosan may be reacted with acrylic acid in water solution. The reaction temperature may be in the range of 20-70° C., and the reaction may be allowed to occur for several days, for example about 2-7 days. The acrylated chitosan product may be purified by adjusting the pH of the reaction mixture to alkaline pH, dialyzing against deionized water and lyophilizing to yield N-acrylated chitosan.

The aCHN according to the present invention forms a flowable solution in water at a pH of less than about 7 and preferably greater than about 6, and gels or solidifies at a physiological pH of about 6.9 to about 7.4. The aCHN of the invention may comprise a range of degrees of polymerization and degrees of substitution without departing from the principles of the invention, but the aCHN of the invention forms a gel or solid at a physiological pH such that the flowability or liquidity of the composition exhibited at a lower pH ceases. A feature of the present invention is that the composition of the invention is flowable at a pH of about 6, such that it may be in contact with living tissue without causing severe corrosive damage such as a composition at a lower pH could, but gels or solidifies at physiological pH such that it may occlude vasculature.

For use as a therapeutic composition, the aCHN solid is dissolved in an aqueous medium, which may be water or saline. A preferred concentration of the aCHN in the aqueous medium is about 1-5% w/v, but higher or lower concentrations may be employed in some cases. Additional components such as buffers, preservatives, stabilizers, surfactants, emulsifiers, nutrients, or dispersants may be present in the composition of the invention.

Bioactive agents may be combined with aCHN solutions by simply blending commercially available solutions of polypeptides or other agents with the aqueous aCHN solutions, with gentle mixing. Cells may likewise be blended with the composition, preferably immediately prior to emplacement to enhance survival of living cells.

A radiopaque material that is optionally incorporated in the composition may be fine particles of a selected radiopaque metal, such as gold, platinum, tantalum or the like.

A bioactive agent incorporated into the composition of the invention may be a regenerative agent such as one or more human growth modulating factors such as interleukins, transformation growth factor-b, fibroblast growth factor or vascular endothelial growth factor; or the agent may be a gene therapy agent, a cogener of platelet derived growth factor, or a monoclonal antibody directed against growth factors; or the agent may be a drug, a cell regeneration factor, drug-producing cells, or regenerative cells.

Due to the abundance of cationic amino groups along the structure of chitosan, it is known that drugs with carboxyl groups can been conjugated thereto and sustained release can be achieved through the hydrolysis of the amide or ester bonds linking drugs to the chitosan molecule. Y. D. Sanzgiri, et al., Pharm. Res., 1, 418 (1990). As a polyelectrolyte, chitosan can also electrostatically conjugate sensitive bioactive agents (e.g., recombinant proteins, such as VEGF) while preserving their bioactivities and enhancing their stabilities. Such derivatives may be formed with the acrylated chitosan of the present invention, and will likewise serve to provide for sustained release and to preserve the bioactivity and to enhance the stability of the conjugated agent(s).

The types of cells that may be incorporated into the composition include progenitor cells of the same type as those from the vascular site, for example an aneurysm, and progenitor cells that are histologically different from those of the vascular site such as embryogenic or adult stem cells, that can act to stabilize the vasculature and/or to accelerate the healing process. The therapeutic composition comprising cells can be administered in the form of a solution or a suspension of the cells mixed with the polymer solution, such that the cells are substantially immobilized within the vascular site upon gelation of the aCHN.

In the case of a vascular site comprising an aneurysm, this serves to concentrate the effect of the therapeutic agent or the cells within the aneurysm and to provide for release of the agent or of the cells or of cellular products over a course of time.

According to a method of the invention, for instance in treatment of an aneurysm, a catheter is maneuvered into position in the parent vessel comprising the aneurysm, and the composition of the invention is delivered endovascularly through the catheter into the aneurysm, where the solution becomes increasingly more viscous and eventually solidifies or gels upon attaining physiological pH after exposure to body fluids. During introduction of the aCHN solution into the aneurysm, it can be imaged by common techniques to allow the physician to monitor the treatment of the aneurysm if the radiopaque material has been added. Once introduced into the aneurysm or the blood vessel to be occluded, the solution gels to block blood flow into the aneurysm or through the vessel. If the composition contains one or more therapeutic agents useful to cause healing of an aneurysm, the agents gradually diffuse and disperse from the gel mass into the aneurysm, to promote the growth of a cellular mass in the void of the aneurysm. If the composition contains cells, the cells themselves may be either released from the gel or products produced by the cell may be released from the gel.

The method of the present invention can be used to embolize normal or abnormal vascular sites. Abnormal vessel sites that can be treated in addition to cerebral aneurysms include aortic aneuryms, arteriovenous malformations, and other vascular defects such as a fistula (an abnormal duct or passage) or a telangiectasia (chronic dilation of a group of capillaries). Afflicted sites on or in normal vasculature can also be located (diagnosed) and/or treated by embolization of vessels, including tumors or other abnormal tissue growth. In the case of an aneurysm, the hydrogel may occlude the entire volume of the aneurysm, as in the case of a fusiform aneurysm or a saccular or berry aneurysm, or the neck of a saccular or berry aneurysm, to reduce the risk of rupture and thrombus formation but allow for continued circulation. In other situations, for example to interrupt the blood supply of a tumor, a more complete blockage of the flow of blood can be achieved. More complete blockage of blood flow may also be employed to prevent downstream hemorrhage, pooling, and other deleterious effects.

The abundance of positive charges on aCHN enables the electrostatic binding of biologically active proteins such as rhVEGF. This is the most gentle mode of conjugating proteins and thus protecting and preserving the bioactivity of sensitive proteins like rhVEGF. The conjugation of proteins like rhVEGF to aCHN also serves as a mechanism for modulating the biological activity of the growth factor, thereby limiting the potential for induction of uncontrolled tissue development.

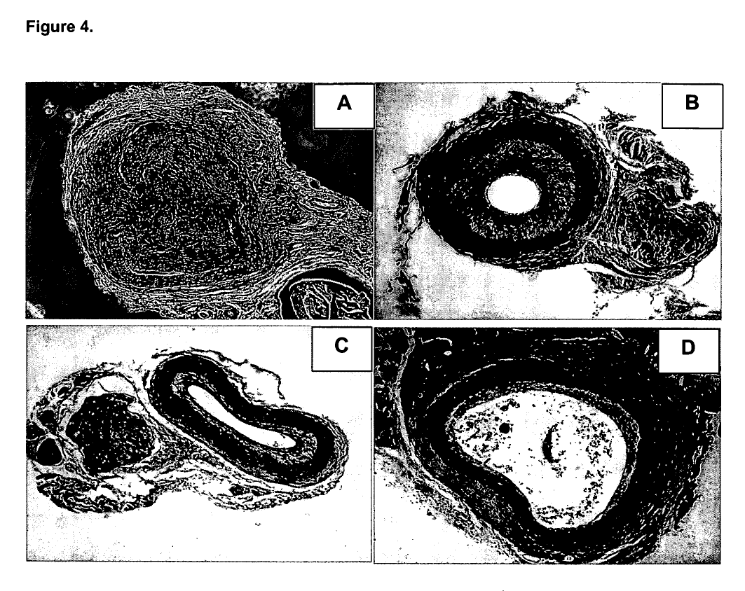

As shown in FIG. 4A, the application of the aCHN-VEGF combination results in a profound response leading to complete filling of the aneursymal sac with fibrous tissue. Interestingly, the application of the aCHN polymer gel alone also resulted in an intense response as indicated by the massive tissue proliferation (FIG. 4B). This effect was likely induced by a combination of inflammatory responses by the presence of aCHN, which induces fibrotic tissue formation, and the stenotic response to arterial injury induced by polymer infusion.

The presence of a stenotic-type response can be substantiated by the moderate tissue proliferation produced by the infusion of saline and VEGF solution (FIGS. 4C & 4D). Nonetheless, the stenotic response alone could not completely account for the profound tissue generation effect of the vessels treated with aCHN alone. Lastly, there was no evidence of angioma development in all the animals treated with rhVEGF. The implication is that the aCHN indeed exerted a certain degree of control on the activity of rhVEGF through electrostatic interaction with its amine groups, thereby, moderating its activity.

The invention will be further described by reference to the following detailed examples wherein both chitosan and acrylic acid were obtained from Sigma-Aldrich (St. Louis, Mo. 63178). The chitosan used was practical grade (>85% deacetylated). The dialysis tubing (MWCO 3,000) was purchased from Spectrum Lab (Racho Dominguez, Calif.). Recombinant human vascular endothelial growth factor (rhVEGF) was obtained from R&D Systems, Minneapolis, Minn. All other chemicals were of reagent grade and distilled and deionized water was used.

### EXAMPLES

### Example 1

Synthesis of Ampholytic Chitosan and Preparation of Bioactive Ampholytic Chitosan Solution

For a typical synthesis, three grams of chitosan was dissolved in 150 ml of 2.75% (v/v) aqueous acrylic acid solution. It was heated and maintained at 50° C. under constant vigorous agitation for 48 hours. Upon cooling to ambient temperature, the pH of the reaction mixture was adjusted to 11 using 1 M NaOH solution. After extensive dialysis for 3 days, the ampholytic chitosan (aCHN) was recovered by lyophilization.

A two percent (w/v) aCHN solution was prepared by dissolving the proper amount of aCHN in water previously adjusted to between pH 6.0 to 6.5. A stock rhVEGF solution (250 ng/μl) was prepared by dissolving rhVEGF in sterile PBS. One hundred microliters of the rhVEGF solution was gently blended with 900 μL of the aCHN solution prepared previously with a micropipette tip to form a bioactive viscous VEGF/aCHN solution.

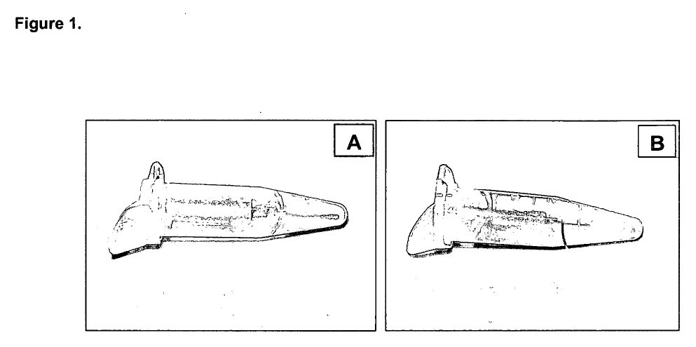

FIG. 1 shows the appearance of an aCHN solution initially (FIG. 1A) and after gelation (FIG. 1B) in the presence of pH 7.4 phosphate buffered saline (PBS). The aCHN solution forms an opaque gel insoluble at physiological pH.

### Example 2

Use of Ampholytic Chitosan to Treat Murine Aneurysm Model

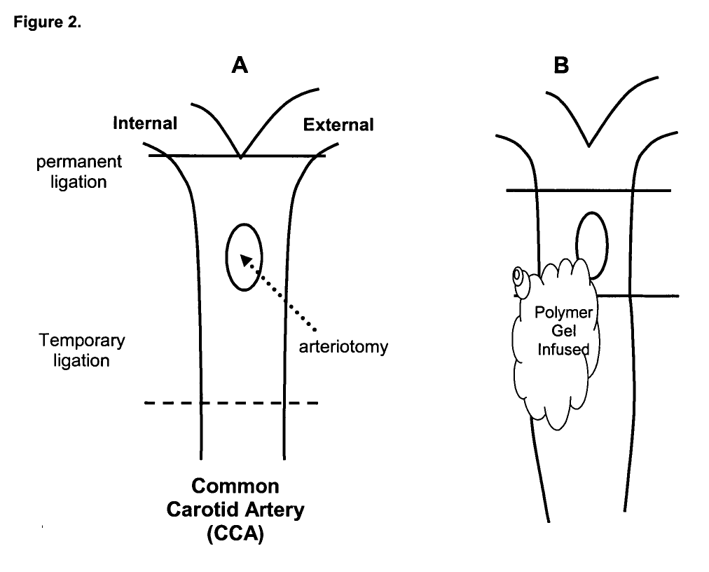

The animal model used was modified from a previously established procedure for adult rats.¹²⁻¹⁷ Sprague-Dawley rats (375 to 450 g) were anesthetized with an intraperitoneal injection of 60 mg/kg sodium pentobarbital and maintained at a temperature of 37° C. throughout the entire procedure. A right paramedian incision was made from the angle of the mandible to the mid-clavicle area. The superficial fascia and muscle layers were separated with blunt dissection until the carotid bundle could be observed. The investing fascia of the common carotid artery (CCA) was incised and the CCA was skeletonized. A permanent ligature was placed proximal to the CCA bifurcation, and a temporary ligature was placed 1 cm distal to the origin of the CCA (FIG. 2). After proximal control of the CCA had been obtained, with complete cessation of arterial blood flow, a small arteriotomy was made 2 mm proximal to the distal ligature. Polymer gel preloaded in a 250 μL Hamilton syringe with a 26-gauge needle was then slowly infused into the CCA. Each animal received a total of 10 μL of the aCHN/VEGF gel (containing a total of 250 ng of VEGF). Likewise, the materials used as controls (aCHN gel, VEGF solution, and saline) were infused into the arteries of the corresponding animals. A new ligature was placed just distal to the arteriotomy, to exclude it from the circulation. The proximal ligature was released to restore blood flow in the CCA segment. Marked vasodilation proximal to the second permanent ligature would occur upon removal of the temporary ligature. The operative field was closed with staples, and the animals were returned to their cages and allowed to recover for two weeks. The animals were administered buphenorphine (0.1-0.5 mg/kg; subcutaneously, daily for 2 days) for pain relief.

Two weeks after the infusion of polymer gel, the rats were euthanized with CO₂. The original incision was reopened and the CCA segment previously infused with polymer gel were resected and preserved in formalin. Following standard histology processing protocols, formalin fixed CCA segments were embedded in paraffin, sectioned, and stained with hematoxylin and eosin. The sections were observed under a microscope (Zeiss Axiovert 200M, Thornwood, N.Y.) and the images were captured and digitized with a camera (AxioCam MRc, Zeiss, Thornwood, N.Y.). The images were analyzed and quantified by the NIH Image J software for their percent occlusion. The data were expressed as mean±standard deviation. Student's t-test was used to determine the statistical differences between groups. Semi-quantitative pathological evaluation on vessel intimal, media and luminal proliferation of the histology sections were performed by a single observer (JMA) who was blinded to the experimental protocol.

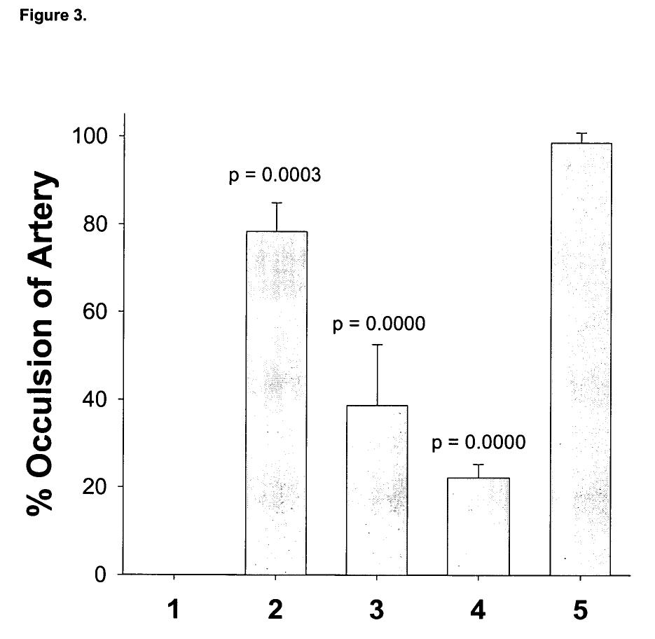

Mean occlusion rates for the vessels are summarized in FIG. 3 with representative histology sections depicted in FIG. 4. As evident from FIG. 4A, the aCHN/VEGF group (n=5) showed virtually complete occlusion of the arterial lumen (98.6±2.2%, FIG. 4A). The aCHN group (n=4) alone showed profound intimal hyperplasia and the lumen was partially filled (78.4±6.5%, FIG. 4B), however, the occlusion was statistically smaller than the aCHN/VEGF group. The saline (n=3) or VEGF solution (n=3) groups showed mild to moderate intimal proliferation response (38.7±13.9% and 22.2±3.1%, respectively, FIGS. 4C and 4D). The control group that received no intervention showed normal appearing vessels (results not shown here). However, there was evidence of vasodilation on gross sectioning of the control vessels.

The results of the pathological scoring for vessel intimal, media and luminal proliferation (all on Grades 0-4) were summarized in Table 1. Comparing scores of the aCHN/VEGF group, results were all significantly greater when compared to other groups (saline, rhVEGF, aCHN). This underscored the advantage of combining the environmentally responsive aCHN gel with VEGF.

TABLE 1
Grading for vessel proliferation. The statistical differences (p-value)
between the group received VEGF-aCHN polymer gel and other
treatments were compared.

| | | Initimal | | Media | | | |
|---|---|---|---|---|---|---|---|
| Treat- | | Prolif- | | Prolif- | | Luminal | |
| ment | N | eration | p | eration | p | Proliferation | p |
| None | 3 | 1.0 ± 0.0 | 0.000 | 1.3 ± 0.6 | 0.000 | 1.0 ± 0.0 | 0.000 |
| Saline | 3 | 2.0 ± 0.0 | 0.000 | 2.3 ± 0.6 | 0.007 | 1.0 ± 0.0 | 0.000 |
| aCHN | 4 | 3.3 ± 0.5 | 0.011 | 3.0 ± 0.0 | 0.010 | 2.3 ± 0.5 | 0.000 |
| polymer | | | | | | | |
| gel | | | | | | | |
| VEGF | 3 | 1.3 ± 0.6 | 0.000 | 1.3 ± 0.6 | 0.000 | 1.0 ± 0.0 | 0.000 |
| solution | | | | | | | |
| VEGF- | 5 | 4.0 ± 0.0 | N/A | 3.8 ± 0.5 | N/A | 4.0 ± 0.0 | N/A |
| aCHN | | | | | | | |
| polymer | | | | | | | |
| gel | | | | | | | |

All the citations listed below are incorporated herein by reference.
1. Byrne J V, Sohn M J, Molyneux A J, Chir B. Five-year experience in using coil embolization for ruptured intracranial aneurysms: outcomes and incidence of late rebleeding. J Neurosurg. 1999; 90:656-63.
2. Friedman J A, Nichols D A, Meyer F B, Pichelmann M A, McIver J I, Toussaint L G 3rd, Axley P L, Brown R D Jr: Guglielmi detachable coil treatment of ruptured saccular cerebral aneurysms: retrospective review of a 10-year single-center experience. AJNR Am J Neuroradiol. 2003; 24:526-533.
3. Gonzalez N, Murayama Y, Nien Y L, Martin N, Frazee J, Duckwiler G, Jahan R, Gobin Y P, Vinuela F. Treatment of unruptured aneurysms with GDCs: clinical experience with 247 aneurysms. AJNR Am J Neuroradiol. 2004; 25:577-583.
4. Kole M K, Pelz D M, Kalapos P, Lee D H, Gulka I B, Lownie S P. Endovascular coil embolization of intracranial aneurysms: important factors related to rates and outcomes of incomplete occlusion. J Neurosurg 2005; 102:607-615.
5. Murayama Y, Vinuela F, Tateshima S, Gonzalez N R, Song J K, Mahdavieh H, Iruela-Arispe L. Cellular responses of bioabsorbable polymeric material and Guglielmi detachable coil in experimental aneurysms. Stroke 2002; 33:1120-1128.
6. Murayama Y, Vinuela F, Tateshima S, Song J K, Gonzalez N R, Wallace M P. Bioabsorbable polymeric material coils for embolization of intracranial aneurysms: a preliminary experimental study. J Neurosurg. 2001; 94:454-463.
7. Murayama Y, Vinuela F, Suzuki Y, Akiba Y, Ulihoa A, Duckwiler G R, Gobin Y P, Vinters H V, Iwaki M, Abe T. Development of the biologically active Guglielmi detachable coil for the treatment of cerebral aneurysms. Part II: an experimental study in a swine aneurysm model. AJNR Am J Neuroradiol. 1999; 20:1992-1999.
8. Kallmes D F, Fujiwara N H, Yuen D, Dai D, Li S T. A collagen-based coil for embolization of saccular aneurysms in a New Zealand White rabbit model. AJNR Am J Neuroradiol. 2003; 24:591-596.
9. Matsumoto H, Terada T, Tsuura M, Itakura T, Ogawa A. Basic fibroblast growth factor released from a platinum coil with a polyvinyl alcohol core enhances cellular proliferation and vascular wall thickness: an in vitro and in vivo study. Neurosurgery 2003; 53:402-407; discussion 407-408.
10. Murayama Y, Vinuela F, Suzuki Y, Do H M, Massoud T F, Guglielmi G, Ji C, Iwaki M, Kusakabe M, Kamio M, Abe T. Ion implantation and protein coating of detachable coils for endovascular treatment of cerebral aneurysms: concepts and preliminary results in swine models. Neurosurgery 1997; 40:1233-1243; discussion 1243-1244.
11. de Gast A N, Altes T A, Marx W F, Do H M, Helm G A, Kallmes D F. Transforming growth factor beta-coated platinum coils for endovascular treatment of aneurysms: an animal study. Neurosurgery 2001; 49:690-694; discussion 694-696.
12. Abrahams J M, Forman M S, Grady M S, Diamond S L. Delivery of human vascular endothelial growth factor with platinum coils enhances wall thickening and coil impregnation in a rat aneurysm model. AJNR Am J Neuroradiol. 2001; 22:1410-1417.
13. Marx W E, Cloft H J, Helm G A, Short J G, Do H M, Jensen M E, Kallmes D E. Endovascular treatment of experimental aneurysms by use of biologically modified embolic devices: coil-mediated intraaneurysmal delivery of fibroblast tissue allografts. AJNR Am J Neuroradiol. 2001; 22:323-33.
14. Murayama Y, Suzuki Y, Vinuela F, Kaibara M, Kurotobi K, Iwaki M, Abe T. Development of a biologically active Guglielmi detachable coil for the treatment of cerebral aneurysms. Part I: in vitro study. AJNR Am J Neuroradiol. 1999; 20: 1986-1991.
15. Kallmes D F, Williams A D, Cloft H J, Lopes M B, Hankins G R, Helm G A. Platinum coil-mediated implantation of growth factor-secreting endovascular tissue grafts: an in vivo study. Radiology 1998; 207:519-523.
16. Abrahams J M, Song C, DeFelice S, Grady M S, Diamond S L, Levy R J. Endovascular microcoil gene delivery using immobilized anti-adenovirus antibody for vector tethering. Stroke 2002; 33:1376-1382.
17. Abrahams J M, Forman M S, Grady M S, Diamond S L. Biodegradable polyglycolide endovascular coils promote wall thickening and drug delivery in a rat aneurysm model. Neurosurgery. 2001; 49:1187-1193; discussion 1193-1195.
18. Dawson R C, Krisht A F, Barrow D L, Joseph G J, Shengelaia G G, Bonner G. Treatment of experimental aneurysms using collagen-coated microcoils. Neurosurgery. 1995; 36:133-139; discussion 139-140.
19. Dawson R C, 3rd, Shengelaia G G, Krisht A F, Bonner G D. Histologic effects of collagen-filled interlocking detachable coils in the ablation of experimental aneurysms in swine. AJNR Am J Neuroradiol. 1996; 17:853-858.
20. Kallmes D F, Borland M K, Cloft H J, Altes T A, Dion J E, Jensen M E, Hankins G R, Helm G A. In vitro proliferation and adhesion of basic fibroblast growth factor-producing fibroblasts on platinum coils. Radiology. 1998; 206:237-243.
21. Kwan E S, Heilman C B, Roth P A. Endovascular packing of carotid bifurcation aneurysm with polyester fiber-coated platinum coils in a rabbit model. AJNR Am J Neuroradiol. 1993; 14:323-33.
22. Tamatani S, Ozawa T, Minakawa T, Takeuchi S, Koike T, Tanaka R. Radiologic and histopathologic evaluation of canine artery occlusion after collagen-coated platinum microcoil delivery. AJNR Am J Neuroradiol. 1999; 20:541-545.
23. Greisler H P. Interactions at the blood/material interface. Ann Vasc Surg. 1990; 4:98-103.
24. Byrne J V, Hope J K, Hubbard N, Morris J H. The nature of thrombosis induced by platinum and tungsten coils in saccular aneurysms. AJNR Am J Neuroradiol. 1997; 18:29-33.
25. Bavinzski G, Talazoglu V, Killer M, Richling B, Gruber A, Gross C E, Plenk H. Gross and microscopic histopathological findings in aneurysms of the human brain treated with Guglielmi detachable coils. J Neurosurg. 1999; 91:284-293
26. Castro E, Fortea F, Villoria F, Lacruz C, Ferreras B, Carrillo R. Long-term histopathologic findings in two cerebral aneurysms embolized with Guglielmi detachable coils. AJNR Am J Neuroradiol. 1999; 20:549-552
27. Horowitz M B, Purdy P D, Burns D, Bellotto D. Scanning electron microscopic findings in a basilar tip aneurysm embolized with Guglielmi detachable coils. AJNR Am J Neuroradiol. 1997; 18:688-690
28. Mizoi K, Yoshimoto T, Takahashi A, Nagamine Y. A pitfall in the surgery of a recurrent aneurysm after coil embolization and its histological observation: technical case report. Neurosurgery 1996; 39:165-169
29. Molyneux A J, Ellison D W, Morris J, Byrne J V. Histological findings in giant aneurysms treated with Guglielmi detachable coils. Rreport of two cases with autopsy correlation. J Neurosurg. 1995; 83:129-132
30. Shimizu S, Kurata A, Takano M, Takagi H, Yamazaki H, Miyasaka Y, Fujii K. Tissue response of a small saccular aneurysm after incomplete occlusion with a Guglielmi detachable coil. AJNR Am J Neuroradiol. 1999; 20:546-548
31. Stiver S I, Porter P J, Willinsky R A, Wallace M C. Acute human histopathology of an intracranial aneurysm treated using Guglielmi detachable coils: case report and review of the literature. Neurosurgery 1998; 43:1203-1208

All publications, patents and patent applications are incorporated herein by reference. While in the foregoing specification this invention has been described in relation to certain preferred embodiments thereof, and many details have been set for purposes of illustration, it will be apparent to those skilled in the art that the invention is susceptible to additional embodiments and that certain of the details described herein may be varied considerably without departing from the basic principles of the invention.

We claim:

1. A method for embolizing a vascular site, comprising introducing into the interior of the vascular site an aqueous solution of an acrylated chitosan such that the solution solidifies or gels in situ to partially or totally fill the vascular site.

2. The method of claim 1 wherein the vascular site is a vascular aneurysm.

3. The method of claim 2 wherein the aneurysm is an intracranial aneurysm.

4. The method of claim 3 wherein the intracranial aneurysm is a anterior circulation aneurysm.

5. The method of claim 3 wherein the intracranial aneurysm is a posterior circulation aneurysm.

6. The method of claim 1 wherein the vascular site is disposed in an artery, vein or lymph duct.

7. The method of claim 1 wherein the vascular site is a normal blood vessel or lymph duct, or an aneurysm, a fistula, an arteriovenous malformation, or a telangiectasia

8. The method of claim 1 wherein the aqueous solution is introduced by means of an endovascular catheter.

9. The method of claim 1 wherein the aqueous solution is adjusted to about pH 6-6.5 prior to introduction.

10. The method of claim 1 wherein the aqueous solution comprises about 1-5 wt-% acrylated chitosan.

11. The method of claim 1 wherein the aqueous solution further comprises an amount of a bioactive agent effective to stimulate cellular growth in said site.

12. The method of claim 11 wherein the agent is VEGF or FGF.

13. The method of claim 12 wherein the agent VEGF or FGF is stabilized with an effective amount of heparin.

14. The method of claim 1 wherein the aqueous solution further comprises a radiopaque material.

15. The method of claim 1 wherein the aqueous solution further comprises intact cells.

16. The method of claim 15 wherein the intact cells are progenitor cells of the same type as cells from the vascular site or progenitor cells that are histologically different from cells from the vascular site.

17. The method of claim 16 wherein the progenitor cells that are histologically different from cells from the vascular site comprise embryogenic or adult stem cells.

18. A therapeutic composition for embolization of a vascular site comprising an effective embolic amount of an acrylated chitosan in combination with a liquid vehicle.

19. The composition of claim 18 wherein the composition is a flowable aqueous solution having a pH of about 6.0 to about 6.5.

20. The composition of claim 19 wherein the composition forms a gel or solid at a pH of about 6.9 to about 7.4.

21. The composition of claim 18 further comprising an amount of an agent effective to stimulate or cause vascular cell growth.

22. The composition of claim 21 wherein the agent is VEGF or FGF.

23. The composition of claim 18 further comprising intact cells.

24. The composition of claim 23 wherein the intact cells are progenitor cells of the same type as cells from the vascular site or progenitor cells that are histologically different from cells from the vascular site.

25. The composition of claim 24 wherein the progenitor cells that are histologically different from cells from the vascular site comprise embryogenic or adult stem cells.

26. The composition of claim 18 wherein a bioactive agent is conjugated to the acrylated chitosan either electrostatically or by formation of amide bonds.

27. A method comprising embolizing a vascular site, comprising introducing the flowable aqueous solution of claim 20 into the site so that a gel or solid is formed in situ, to provide partial or complete occlusion.

28. The method of claim 27 further comprising treatment of the vascular site wherein the composition includes a bioactive agent or intact cells.

29. The method of claim 27 wherein the bioactive agent is VEGF or FGF.

30. The method of claim 27 wherein the intact cells are progenitor cells of the same type as cells from the vascular site or progenitor cells that are histologically different from cells from the vascular site.

31. The method of claim 27 wherein the progenitor cells that are histologically different from cells from the vascular site comprise embryogenic or adult stem cells.
